(19) United States
(12) Patent Application Publication
(10) Pub. No.: US 2006/0172436 A1
(43) Pub. Date: Aug. 3, 2006

(54) Diagnosis supporting system

(75) Inventors: Wataru Hattori, Tokyo (JP); Toru Sano, Tokyo (JP); Masakazu Baba, Tokyo (JP); Kazuhiro Iida, Tokyo (JP); Hisao Kawaura, Tokyo (JP); Noriyuki Iguchi, Tokyo (JP); Hiroko Someya, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: NEC Corporation

(21) Appl. No.: 10/549,116

(22) PCT Filed: Feb. 26, 2004
(86) PCT No.: PCT/JP04/02244

§ 371 Date: Sep. 14, 2005

(30) Foreign Application Priority Data
Mar. 14, 2003 (JP) 2003-069822

(51) Int. Cl. G01N 33/543; G06F 19/00

(52) U.S. Cl. 436/518; 702/19

(57) ABSTRACT

A diagnosis supporting system (10) includes a diagnosis data obtaining unit (20) that obtains diagnosis data in which a movement parameter reflecting a movement speed of each component in separating a sample collected from a test subject into plural components with a chip (12) and a character of each component is in correspondence, a parameter storing unit (34) that stores the movement parameter of a characteristic component showing a state of suffering from a specific disease in correspondence with the disease, a relationship data storing unit (35) that stores relationship data showing a relationship between the character of the characteristic component and a possibility of suffering from a specific disease, a detecting unit (21) that detects the characteristic component from the diagnosis data based on the movement parameter of the characteristic component and the movement parameter of the diagnosis data by making reference to the parameter storing unit (34), and an inference processing unit (22) that infers the possibility that the test subject is suffering by making reference to the relationship data storing unit (35) based on the character of the detected characteristic component.

### TECHNICAL FIELD

The present invention relates to a diagnosis supporting system that infers the possibility that a test subject is suffering from a disease based on a sample collected from the test subject.

### BACKGROUND ART

Conventionally, a method is known for carrying out functional analysis such as expression and interaction of protein or identification or the like by peptide mapping with the use of a protein chip (for example, Patent Document 1). For the protein chip, peptides of known and different kinds of structures are fixed in a matrix form on a substrate such as a slide glass. For the peptides fixed to the substrate, a substance is selected that has an affinity to a protein (protein marker) that expresses itself by a certain specific disease, and specifically interacts to adsorb and capture the protein marker to bond to the substrate. When a sample is added to such a protein chip and incubated, substances that interact with the substance fixed to the substrate are bonded to the substrate. In this state, the protein chip is washed with a buffer or the like to remove the components that are not bonded to the substrate and do not have an affinity. Subsequently, the protein chip is tested with a mass analyzing apparatus or a fluorescence detecting apparatus, whereby the expression of the protein marker can be detected. By observing the interaction with various peptides with the use of this, the presence or absence of a protein marker that expresses itself by a certain specific disease can be determined. Patent Document 1: Japanese Patent Application Laid-Open (JP-A) No. 2002-365288

### DISCLOSURE OF THE INVENTION

However, with a conventional protein chip, in order to detect a protein marker, which is a proper protein that expresses itself by a specific disease, it was necessary to fix a proper substance that has an affinity to each protein marker, to a substrate. For this reason, the protein chip has been one proper to the disease. Therefore, a different chip is needed for each disease, so that the chip has a narrow range of usability. When one wishes to determine the presence or absence of a protein marker for plural diseases, it was necessary to prepare a plurality of chips, thereby making the preparation a cumbersome one.

The present invention has been made in view of the above circumstances, and an object of the present invention is to provide a system that performs diagnosis support for various diseases in a simple manner and for wide use.

According to the present invention, there is provided a diagnosis supporting system that infers a possibility that a test subject is suffering from a disease based on a sample collected from the test subject, the diagnosis supporting system including: a diagnosis data obtaining unit that obtains diagnosis data in which a movement parameter reflecting a movement speed of each component in letting the sample move in a predetermined region and separating the sample into plural components in accordance with a difference of the movement speed and a character of each component are in correspondence; a parameter storing unit that stores the movement parameter of a characteristic component characteristically showing a state of suffering from a specific disease in correspondence with the disease; a relationship data storing unit that stores relationship data showing a relationship between the character of the characteristic component and a possibility of suffering from the specific disease; a detecting unit that reads the movement parameter of the characteristic component out from the parameter storing unit and detects the characteristic component from the diagnosis data based on the movement parameter and the movement parameter of the diagnosis data; and an inference processing unit that reads the relationship data out from the relationship data storing unit and infers the possibility that the test subject is suffering from the specific disease based on the character of the characteristic component of the diagnosis data by making reference to the relationship data.

Here, the movement parameter is an amount of movement of each component in a predetermined period of time or a period of time of movement in a predetermined distance. According to the diagnosis supporting system of the present invention, a characteristic component is detected from among the obtained diagnosis data in accordance with a disease constituting an object of diagnosis, and the possibility that the test subject is suffering from the disease is inferred based on the character of the characteristic component, so that the separation of the sample before obtaining the diagnosis data can be carried out in a similar manner irrespective of the kind of the disease constituting the object of diagnosis. This allows the test subject to receive an inference of the possibility of suffering from various diseases easily without the need for preparing different separation means for each disease constituting an object of diagnosis. The diagnosis supporting system of the present invention can be used for an inference of the possibility of suffering of human beings and animals.

Here, the predetermined region can be a flow passageway for separation that is disposed on a chip. A sample collected from a test subject can be separated into plural components with the use of a chip including a flow passageway for separation. In this case, a desired protein marker is not adsorbed and captured with the use of a proper substance having an affinity, but a desired characteristic component is specified and the character thereof is detected by letting the sample flow in the flow passageway for separation to separate the sample into plural components and making the information on each component correspond to the movement parameter, so that the chip for separation of the sample is not dependent on a specific disease, and the detection of various diseases can be carried out with one kind of a chip.

According to the diagnosis supporting system of the present invention, after the diagnosis data are obtained, a proper process is carried out for each disease based on the diagnosis data to infer the possibility of suffering, so that the separation means such as a chip for separation of the sample can be used in common for various diseases. This can enhance the general usability of supporting the diagnosis. Also, when one diagnosis data is obtained, the possibility of suffering from various diseases can be inferred based on that, so that the diagnosis can be supported speedily by simplifying the process.

In the diagnosis supporting system of the present invention, the parameter storing unit can store a plurality of the movement parameters respectively in correspondence with the plural components, in correspondence with the disease, and the detecting unit can detect the characteristic component based on a mutual relationship of the plurality of movement parameters. By this, the characteristic component can be detected using the movement parameters of the plural components as a standard, so that the possibility of suffering can be inferred speedily and with high precision.

In the diagnosis supporting system of the present invention, the parameter storing unit can store also the movement parameter of a marker component that is detected irrespective of suffering from the specific disease, in correspondence with the disease, and the detecting unit can detect a corresponding marker component from the diagnosis data by making reference to the movement parameter of the marker component and determine whether the character of the marker component is appropriate or not and, when it is appropriate, detect the characteristic component and, when it is not appropriate, prompt a user to obtain a sample again. By this, the possibility of suffering can be inferred speedily and with high precision.

In the diagnosis supporting system of the present invention, the parameter storing unit can store the movement parameter of the characteristic component for each of a plurality of diseases, and the detecting unit can, for each disease, read the movement parameter of a corresponding characteristic component out from the parameter storing unit, and respectively detect the characteristic component from the diagnosis data by making reference to the movement parameter.

In the diagnosis supporting system of the present invention, the parameter storing unit can store the movement parameter of the characteristic component for each of a plurality of diseases, the diagnosis data obtaining unit can receive selection of a disease as an object of diagnosis together with the diagnosis data, and the detecting unit can, in accordance with the selection of a disease received by the diagnosis data obtaining unit, read the movement parameter of a corresponding characteristic component out from the parameter storing unit, and detect the characteristic component from the diagnosis data by making reference to the movement parameter.

In the diagnosis supporting system of the present invention, the character can be an amount of change of light when light of a predetermined wavelength is radiated onto each of the separated components. Here, the amount of change of light is represented by any of a wavelength, a reflection angle, an amount of reflection, an amount of transmittance, and an amount of absorption, and a combination of these.

According to the present invention, there is provided a diagnosis supporting system that infers a possibility that a test subject is suffering from a disease based on a sample collected from the test subject, the diagnosis supporting system characterized by comprising: a diagnosis data obtaining unit that obtains diagnosis data in which a movement parameter reflecting a movement speed of each component in letting the sample move in a predetermined region and separating the sample into plural components in accordance with a difference of the movement speed, a nature parameter showing nature of each component in classifying each component further into plural components in accordance with the nature, and a character of each component are in correspondence; a parameter storing unit that stores the movement parameter and the nature parameter of a characteristic component characteristically showing a state of suffering from a specific disease in correspondence with the disease; a relationship data storing unit that stores relationship data showing a relationship between the character of the characteristic component and a possibility of suffering from the specific disease; a detecting unit that reads the movement parameter and the nature parameter of the characteristic component out from the parameter storing unit and detects the characteristic component from the diagnosis data based on these parameters and the movement parameter and the nature parameter of the diagnosis data; and an inference processing unit that reads the relationship data out from the relationship data storing unit and infers the possibility that the test subject is suffering from the specific disease based on the character of the characteristic component of the diagnosis data by making reference to the relationship data.

In this way, classifying the sample into parts in accordance with the difference of movement speed in a predetermined region and the nature for comparison with the relationship data, the possibility of suffering from a disease can be inferred with higher precision. Also, the possibility of suffering from a larger number of diseases can be inferred based on one diagnosis data.

In the diagnosis supporting system of the present invention, the parameter storing unit can store a plurality of the movement parameters and the nature parameter respectively in correspondence with the plural components, in correspondence with the disease, and the detecting unit can detect the characteristic component from the diagnosis data based on a mutual relationship of the plurality of movement parameters and the nature parameter.

In the diagnosis supporting system of the present invention, the parameter storing unit can store the movement parameter and the nature parameter of the characteristic component for each of a plurality of diseases, and the detecting unit can, for each disease, read the movement parameter and the nature parameter out from the parameter storing unit, and respectively detect the characteristic component from the diagnosis data by making reference to the movement parameter and the nature parameter.

In the diagnosis supporting system of the present invention, the parameter storing unit can store the movement parameter and the reference parameter of the characteristic component for each of a plurality of diseases, the diagnosis data obtaining unit can receive selection of a disease as an object of diagnosis together with the diagnosis data, and the detecting unit can, in accordance with the selection of a disease received by the diagnosis data obtaining unit, read the movement parameter and the nature parameter of a corresponding characteristic component out from the parameter storing unit, and detect the characteristic component from the diagnosis data by making reference to the movement parameter and the nature parameter.

In the diagnosis supporting system of the present invention, the relationship data storing unit can store the relationship data for each of a plurality of diseases, and the inference processing unit can, for each disease, read the relationship data out from the relationship data storing unit and infer the possibility of the test subject's suffering from the specific disease by making reference to the relationship data.

In the diagnosis supporting system of the present invention, the relationship data storing unit can store the relationship data of the characteristic component for each of a plurality of diseases, and the inference processing unit can, in accordance with the selection of a disease received by the diagnosis data obtaining unit, read the relationship data of a corresponding disease and infer the possibility of the test subject's suffering from the specific disease by making reference to the relationship data.

In the diagnosis supporting system of the present invention, the character can be a data value showing an amount of presence of a specific substance in the component, and the relationship data storing unit can store relationship data showing a relationship between a character function having this data value as a variable and the possibility of suffering.

The diagnosis supporting system of the present invention can further include a procedure storing unit that stores a procedure of obtaining the diagnosis data for each of a plurality of diseases, and the diagnosis data obtaining unit can receive selection of a disease as an object of diagnosis before obtaining the diagnosis data, and can read the obtaining procedure related to a corresponding disease in accordance with the selection out from the procedure storing unit for presentation. By doing so, the diagnosis data can be obtained by a suitable obtaining procedure in accordance with various diseases even with the use of a chip or separation means of the same kind, so that the possibility of suffering from a disease can be inferred with high precision. Also, by presenting the obtaining procedure to the user, the reproducibility of the procedure for obtaining the diagnosis data can be enhanced. Also, by this, the possibility of suffering from a disease can be inferred with high precision.

The diagnosis supporting system of the present invention can further include: a diagnosis data storing unit that stores the diagnosis data obtained by the diagnosis data obtaining unit in correspondence with a management number; an inference result reading unit that outputs an inference result by the inference processing unit in correspondence with the management number; a doctor diagnosis result receiving unit that receives a diagnosis result of a doctor on a specific disease together with the management number; and a relationship data renewing unit that reads the corresponding diagnosis data out from the diagnosis data storing unit using the management number as a key, and renews the relationship data storing unit by making reference to the character of the characteristic component of the corresponding diagnosis data and the diagnosis result of the doctor. By this, the relationship data storing unit can be renewed by using the management number as a key and making reference also to the diagnosis result of a doctor, so that the precision of the subsequent inference of the possibility of suffering of the diagnosis data can be enhanced.

According to the present invention, the separation means itself such as a chip for separation of a sample is used in common for the inference of the possibility of suffering from plural diseases, and the process of the diagnosis supporting system is made different suitably in accordance with the disease, so that the possibility of suffering from various diseases can be inferred with a high general usability.

### BRIEF DESCRIPTION OF THE DRAWINGS

The aforesaid objects and other objects, features, and advantages will be made more apparent from the preferable embodiments described hereafter and the following drawings associated therewith.

FIG. 1 is a block diagram showing a structure of a diagnosis supporting system in an embodiment of the present invention.

FIG. 2 is a view showing the chip shown in FIG. 1.

FIG. 3 is a view showing one example of the diagnosis data obtained by the diagnosis data obtaining unit.

FIG. 4 is a view showing one example of a data structure of a parameter storing unit.

FIG. 5 is a view in which the diagnosis data shown in FIG. 3 are made into numerical values.

FIG. 6 is a view showing one example of a data structure of a relationship data storing unit.

FIG. 7 is a view showing one example of a data structure of an inference result storing unit.

FIG. 8 is a view showing another example of a data structure of the relationship data storing unit.

FIG. 9 is a view showing still another example of a data structure of the relationship data storing unit.

FIG. 10 is a block diagram showing a diagnosis supporting system in an embodiment of the present invention.

FIG. 11 is a flowchart showing a processing procedure in a measurement side system, an inference processing system, and a hospital system in an embodiment of the present invention.

FIG. 12 is a block diagram showing a diagnosis supporting system in an embodiment of the present invention.

FIG. 13 is a block diagram showing a diagnosis supporting system in an embodiment of the present invention.

FIG. 14 is a perspective view showing a chip and a measuring unit in an embodiment of the present invention.

FIG. 15 is a view showing one example of the diagnosis data obtained by the diagnosis data obtaining unit.

### BEST MODE FOR CARRYING OUT THE INVENTION

### First Embodiment

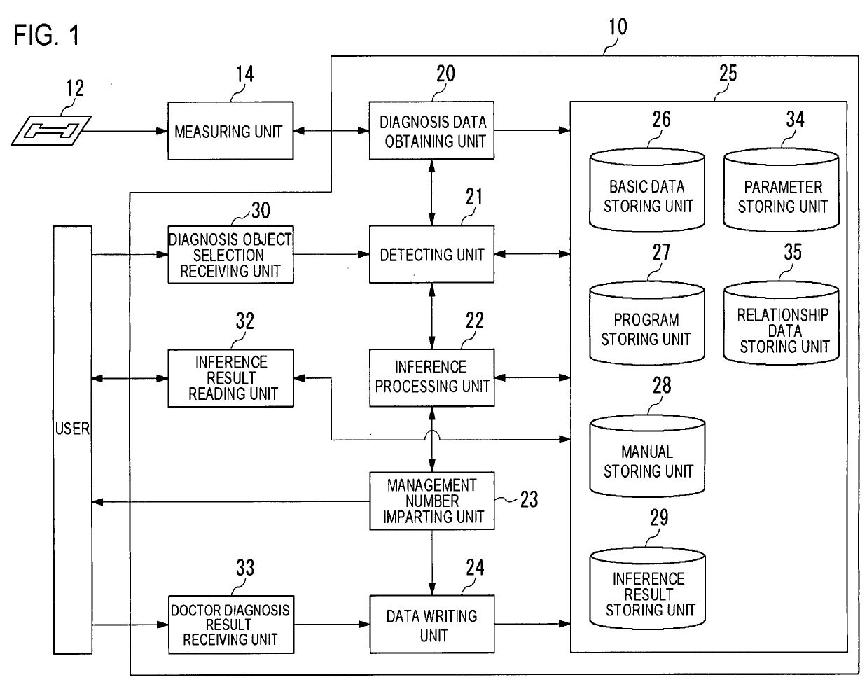

FIG. 1 is a block diagram showing a structure of a diagnosis supporting system 10 in this embodiment.

The diagnosis supporting system 10 according to this embodiment infers a possibility (disease possibility) that a test subject is suffering from a disease based on a sample such as a blood collected from the test subject.

The diagnosis supporting system 10 has a diagnosis data obtaining unit 20, a detecting unit 21, an inference processing unit 22, a management number imparting unit 23, a data writing unit 24, a database 25, a diagnosis object selection receiving unit 30, an inference result reading unit 32, and a doctor diagnosis result receiving unit 33.

A chip 12 includes a flow passageway for separation through which a sample moves, and separates the sample into plural components in accordance with the difference in the molecular size, the molecular weight, the isoelectric point (pH), or the like. A measuring unit 14 measures the character of a component in the sample separated by the chip 12. In this embodiment, the measuring unit 14 is an UV spectroanalysis device.

The diagnosis data obtaining unit 20 obtains diagnosis data in which the character of each component measured by the measuring unit 14 and the movement parameter reflecting the movement speed when each component is separated in accordance with the difference of the movement speed in the flow passageway for separation are in correspondence. Here, the character of a component is the absorptivity when light of a predetermined wavelength is radiated onto each component by the measuring unit 14. Also, the movement parameter is an amount of movement of each component in a constant period of time.

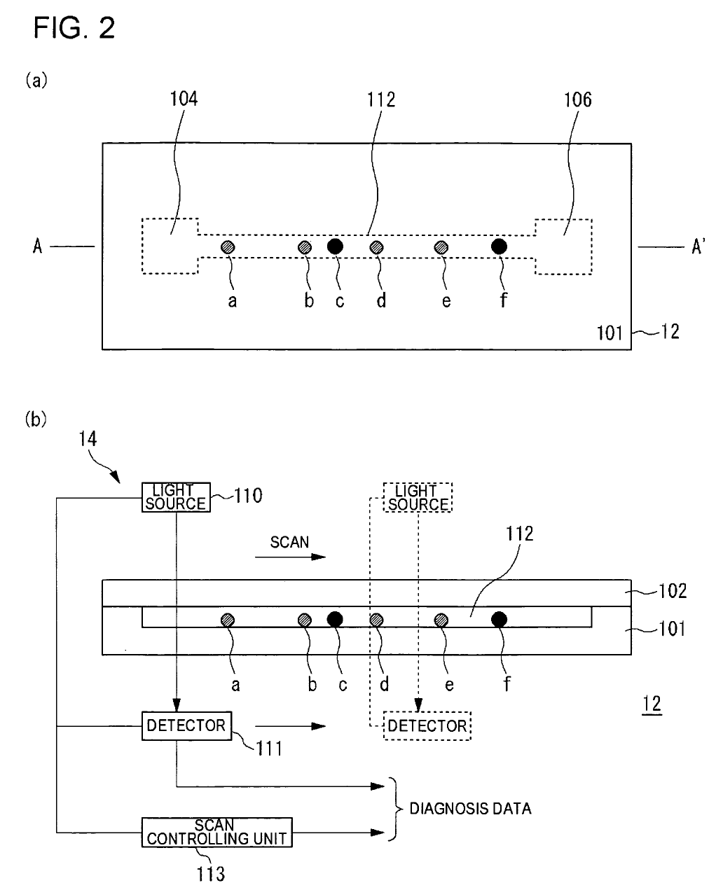

FIG. 2 is a view showing the chip 12. FIG. 2(a) shows a top view of the chip 12. The chip 12 includes a sample introducing unit 104, a flow passageway for separation 112, and a sample collecting unit 106 that are formed on a substrate 101. The chip 12 is not limited to the structure shown in FIG. 2, and may have any structure.

Although not illustrated, in the chip 12, an electrode, for example, can be disposed in the sample introducing unit 104 and in the sample collecting unit 106. When a sample is introduced from the sample introducing unit 104 and a voltage is applied between the electrodes disposed in the sample introducing unit 104 and in the sample collecting unit 106, the components in the sample move towards the sample collecting unit 106 at their respective proper speeds in accordance with the nature such as molecular weight. When the voltage is applied for a constant period of time, the components are separated on the flow passageway for separation 112, as illustrated. Here, from the side close to the sample introducing unit 104, the component a, the component b, the component c, the component d, the component e, and the component f are separated.

FIG. 2(b) is a model view showing an A-A′ cross section of the chip 12 shown in FIG. 2(a) and the measuring unit 14. A cover member 102 is disposed on the substrate 101, and the flow passageway for separation 112 is filled with a solvent. The measuring unit 14 has a light source 110, a detector 111, and a scan controlling unit 113. The scan controlling unit 113 moves the light source 110 and the detector 111 relative to the chip 12. In such a state, light of a predetermined wavelength is radiated from the light source 110; the light source 110 is scanned along the flow passageway for separation 112; and the absorptivity of the light transmitted through each component is detected with the detector 111. The diagnosis data obtaining unit 20 (see FIG. 1) obtains the absorptivity of each component measured by the measuring unit 14, in correspondence with the amount of movement of each component in the flow passageway for separation 112. The amount of movement can be detected based on the amount by which the scan controlling unit 113 moves the detector 111. Also, the measuring unit 14 may scan each component with light of wavelengths within a predetermined range and measure the absorptivity of the light transmitted through each component. In this case, the diagnosis data obtaining unit 20 may obtain absorption spectrum data as a character for each component, and can obtain the absorptivity of the light at a specific wavelength from the absorption spectrum data as a character.

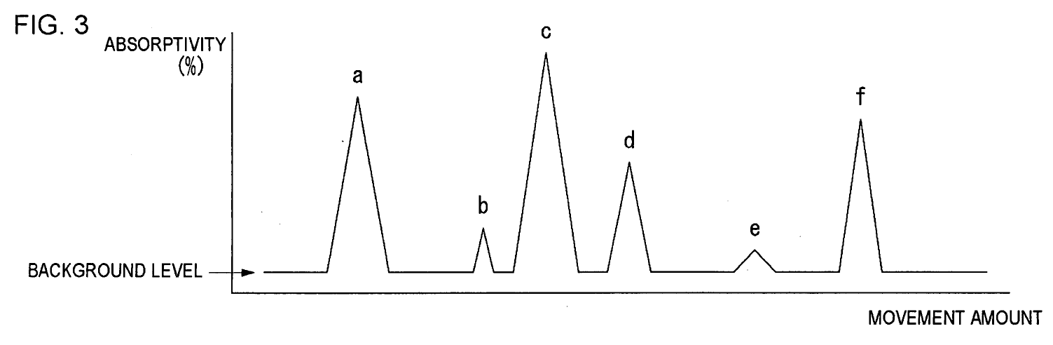

FIG. 3 is a view showing one example of the diagnosis data obtained by the diagnosis data obtaining unit 20. Here, the relationship between the absorptivity and the amount of movement is shown when the light from the light source 110 shown in FIG. 2 is used for scanning to radiate the light along the flow passageway for separation 112 of the chip 12.

Returning back to FIG. 1, the diagnosis data obtaining unit 20 can control the measuring unit 14 so that appropriate diagnosis data on a specific disease can be obtained. In this case, the diagnosis data obtaining unit 20, for example, suitably sets the wavelength of the light radiated from the light source 110 (see FIG. 2) of the measuring unit 14. When the sample is a protein, the light absorptivity of each component is measured by using the light having a wavelength of λ=280 nm, for example.

The detecting unit 21 detects a characteristic component for use in inferring the possibility that the test subject is suffering from a disease among the diagnosis data obtained by the diagnosis data obtaining unit 20. When the characteristic component cannot be detected, the detecting unit 21 can determine that the measurement is impossible or set the measured value to be 0%.

Based on the character of the characteristic component detected by the detecting unit 21, the inference processing unit 22 refers to the database 25 and infers the possibility that the test subject, from which the sample is collected, is suffering from a specific disease.

The management number imparting unit 23 imparts a management number in correspondence with the diagnosis data. The data writing unit 24 stores various data into the database 25.

The database 25 includes a basic data storing unit 26, a program storing unit 27, a manual storing unit 28, an inference result storing unit 29, a parameter storing unit 34, and a relationship data storing unit 35.

The parameter storing unit 34, for each of the plural diseases, stores the movement parameters of plural components constituting an index in detecting the characteristic components from the diagnosis data for use in inferring the possibility that the test subject is suffering from the respective disease. The parameter storing unit 34 stores, for example, the movement parameters of a characteristic component characteristically showing a state of suffering from the respective disease and of a marker component that expresses itself irrespective of suffering from the disease, in correspondence with each of the diseases. The marker component can be a marker agent that is added separately from the sample collected from the test subject. The marker agent is preferably a substance having a high reproducibility of the movement parameter and having specified components, such as fine gold particles, polystyrene beads, or semiconductor quantum dots, for example. In this case, after a marker agent is added to the sample collected from the test subject, the components in the sample are separated on the chip 12. The detecting unit 21 reads the movement parameters of these components out from the parameter storing unit 34, and detects the characteristic component from the diagnosis data based on the read movement parameters and the movement parameters of the diagnosis data.

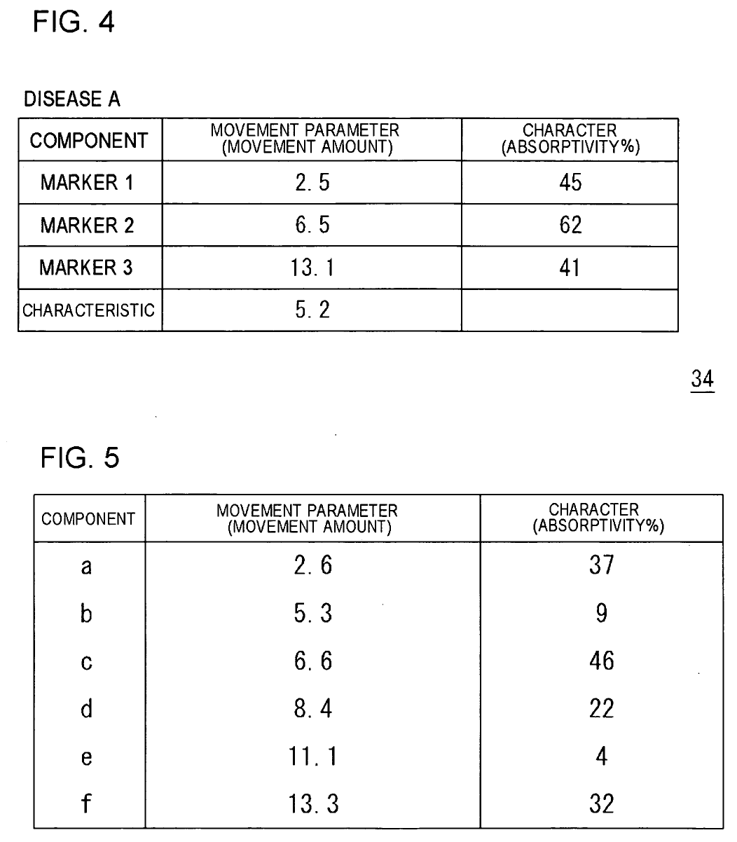

FIG. 4 is a view showing one example of a data structure of the parameter storing unit 34. Here, the movement parameter (amount of movement) is stored for each of the plural marker components 1 to 3 and the characteristic component related to the disease A. For the marker components 1 to 3, the character (absorptivity (%)) is also stored. FIG. 5 is a view in which the diagnosis data shown in FIG. 3 are made into numerical values. The detecting unit 21 reads the movement parameters of the marker components from the parameter storing unit 34 and compares the read movement parameters with the movement parameters of the components a to f of the diagnosis data to detect corresponding marker components from among the components a to f of the diagnosis data. The detecting unit 21 can detect corresponding marker components from the diagnosis data based on the mutual relationship of the movement parameters of the plural marker components. Also, the detecting unit 21 can detect corresponding marker components from the diagnosis data by making reference also to the character of the marker components. Here, the component a, the component c, and the component f are respectively detected as the marker components 1 to 3. Subsequently, the detecting unit 21 detects a characteristic component from the diagnosis data based on the mutual relationship with the movement parameters of these marker components 1 to 3. Here, the component b is detected as the characteristic component.

Returning back to FIG. 1, the relationship data storing unit 35 stores, for each of the plural diseases, the relationship between the character of the above-mentioned characteristic component and the possibility of suffering from the respective disease. In this embodiment, the relationship data storing unit 35 stores relationship data showing a relationship between a character function having the data value showing the character of the characteristic component as a variable and the possibility of suffering from the respective disease. Here, the data value is the absorptivity. For example, the character function is a relative intensity ratio obtained by dividing the absorptivity of the characteristic component with the absorptivity of another component. The relationship data storing unit 35 can store the character function.

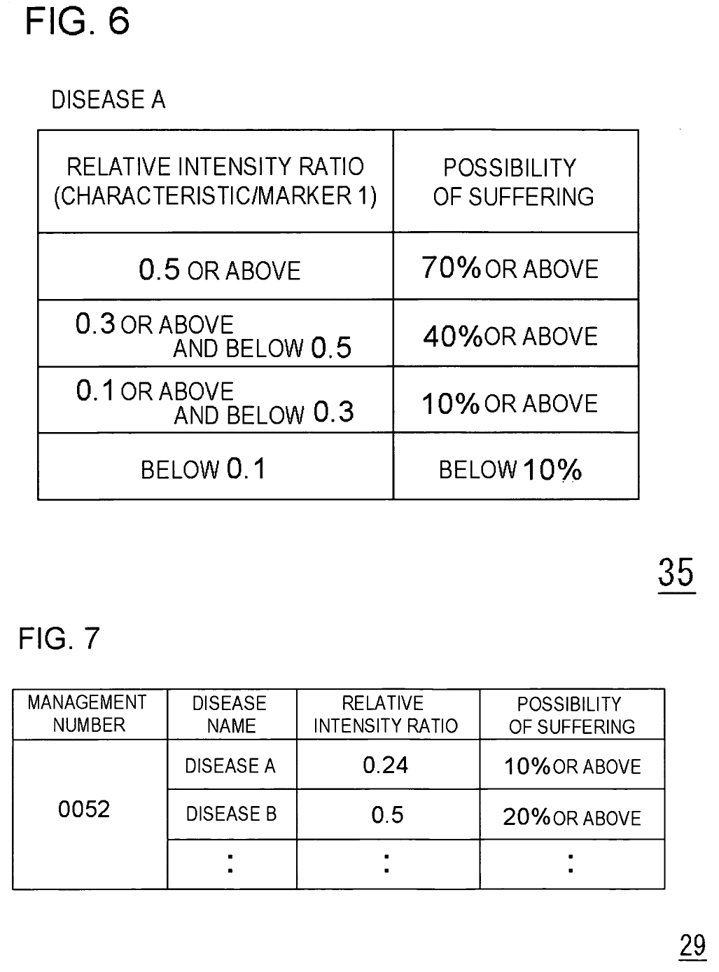

FIG. 6 is a view showing one example of a data structure of the relationship data storing unit 35. Here, the relationship between the relative intensity ratio and the possibility of suffering related to the disease A is stored. Here, the relative intensity ratio can be calculated by dividing the absorptivity of the characteristic component shown in FIG. 4 with the absorptivity of the marker component 1. For example, it is stored that, when the relative intensity ratio is 0.5 or above, the possibility of suffering is 70% or above; when the relative intensity ratio is 0.3 or above and below 0.5, the possibility of suffering is 40% or above; when the relative intensity ratio is 0.1 or above and below 0.3, the possibility of suffering is 10% or above; and when the relative intensity ratio is below 0.1, the possibility of suffering is below 10%.

For example, in the example described above with reference to FIGS. 4 and 5, the component b is detected as the characteristic component, and the component a is detected as the marker component 1, so that the relative intensity ratio can be calculated by (absorptivity of component b)/(absorptivity of component a). Here, from the absorptivity shown in FIG. 5, the relative intensity ratio will be 9/37=0.24. In this case, the inference processing unit 22 infers that the possibility of suffering from the disease A is 10% or above.

Returning back to FIG. 1, the basic data storing unit 26 stores basic data in which the movement parameter of each component and the character are in correspondence, for plural samples. The data writing unit 24 can store the diagnosis data obtained by the diagnosis data obtaining unit 20 in correspondence with the management number as the basic data in the basic data storing unit 26. By this, the basic data can be accumulated successively in the basic data storing unit 26. The relationship between the character stored in the relationship data storing unit 35 and the possibility of suffering can be calculated based on the plural basic data stored in the basic data storing unit 26.

The program storing unit 27 stores, for example, various programs such as a procedure and a program by which the detecting unit 21 detects the components and an analyzing program that defines the procedure by which the inference processing unit 22 infers the possibility of suffering, respectively for plural diseases. Also, the program storing unit 27 can also store a program by which the diagnosis data obtaining unit 20 controls the measuring unit 14.

The manual storing unit 28 stores manuals such as a procedure of obtaining the diagnosis data. The obtaining procedure includes a procedure of preparing samples such as a method of collecting a sample, a method of adjusting the concentration, and a method of using markers, and a procedure of measuring the sample such as the measurement wavelength of the sample. The diagnosis data obtaining unit 20 presents these obtaining procedures to the user. The manual storing unit 28 can store these manuals for each of the diseases.

The inference result storing unit 29 stores the inference result inferred by the inference processing unit 22 in correspondence with the management number. By this, the user can read the inference result out using the management number as a key. FIG. 7 is a view showing one example of a data structure of the inference result storing unit 29. Here, for plural diseases, the relative intensity ratio and the possibility of suffering are stored in correspondence with the management number. For example, the diagnosis data of the management number 0052 are stored in such a manner that the relative intensity ratio related to the disease A is 0.24 and the possibility of suffering is 10% or above, while the relative intensity ratio related to the disease B is 0.5 and the possibility of suffering is 20% or above.

Returning back to FIG. 1, the diagnosis object selection receiving unit 30 receives selection of a disease as an object of diagnosis from the user of the diagnosis supporting system 10. Before obtaining the diagnosis data, the diagnosis data obtaining unit 20 reads the obtaining procedure related to the corresponding disease in correspondence with the selection of a disease received by the diagnosis object selection receiving unit 30 out from the manual storing unit 28, for presentation to the user.

The inference result reading unit 32 receives a management number from the user, and reads the corresponding inference result from the inference result storing unit 29 out using the management number as a key, for presentation to the user. The presentation to the user may be carried out, for example, by displaying the inference result on a monitor or by outputting the inference result with a printer or the like. Although not illustrated, the diagnosis supporting system 10 may have a user identification function, and the management number imparting unit 23 can impart a user ID and a password together with the management number. In this case, the inference result reading unit 32 may present the inference result to the user after carrying out the user identification.

When the test subject has received a diagnosis of a doctor in a hospital, the doctor diagnosis result receiving unit 33 receives the diagnosis result of the doctor in correspondence with the management number. The data writing unit 24 stores the diagnosis data stored in the basic data storing unit 26 in correspondence with the diagnosis result of the doctor based on the management number received by the doctor diagnosis result receiving unit 33. This can enhance the effectiveness of the diagnosis data stored in the basic data storing unit 26. Also, the data writing unit 24 can read a corresponding inference result from the inference result storing unit 29 out using the management number as a key, and suitably renew the relationship data of the relationship data storing unit 35 by making reference to the relative intensity ratio of the inference result and the doctor diagnosis result. This can enhance the precision of the relationship data of the relationship data storing unit 35.

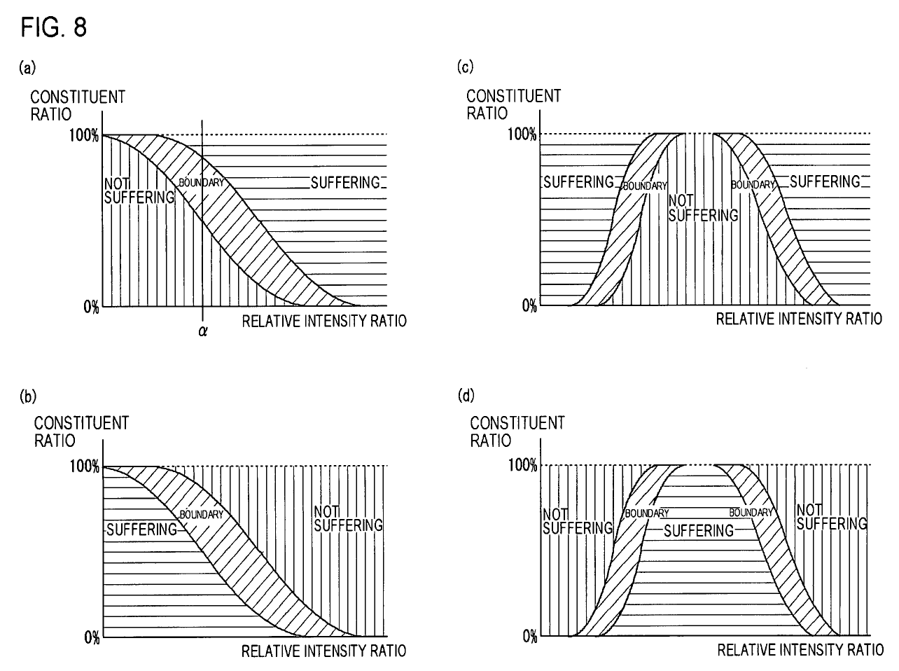

FIG. 8 is a view showing another example of a data structure of the relationship data storing unit 35. Here, the relative intensity ratio, the non-suffering and suffering of the diagnosis data at each relative intensity ratio, and the constituent ratio of the boundary of these are stored based on the plural basic data stored in the basic data storing unit 26. Here, the non-suffering indicates a state of being diagnosed to be not suffering, and the suffering indicates a state of being diagnosed to be suffering. The relationship between the relative intensity ratio and the constituent ratio shows a different pattern depending on the nature of the components contained in the character function and various factors. For example, it may be classified into patterns such as shown in FIGS. 8(a) to 8(d).

In the pattern shown in FIG. 8(a), the possibility of not suffering is extremely high when the relative intensity ratio is almost zero, and the possibility of suffering increases according as the relative intensity ratio gets higher. On the other hand, in the pattern shown in FIG. 8(b), the possibility of suffering is extremely high when the relative intensity ratio is almost zero, and the possibility of not suffering increases according as the relative intensity ratio gets higher. In the pattern shown in FIG. 8(c), the possibility of suffering is extremely high when the relative intensity ratio is almost zero; and the possibility of not suffering increases according as the relative intensity ratio gets higher; and, when the relative intensity ratio exceeds a certain point, the possibility of suffering increases again according as the relative intensity ratio gets higher. In the pattern shown in FIG. 8(d), the possibility of not suffering is extremely high when the relative intensity ratio is almost zero; and the possibility of suffering increases according as the relative intensity ratio gets higher; and, when the relative intensity ratio exceeds a certain point, the possibility of not suffering increases again according as the relative intensity ratio gets higher.

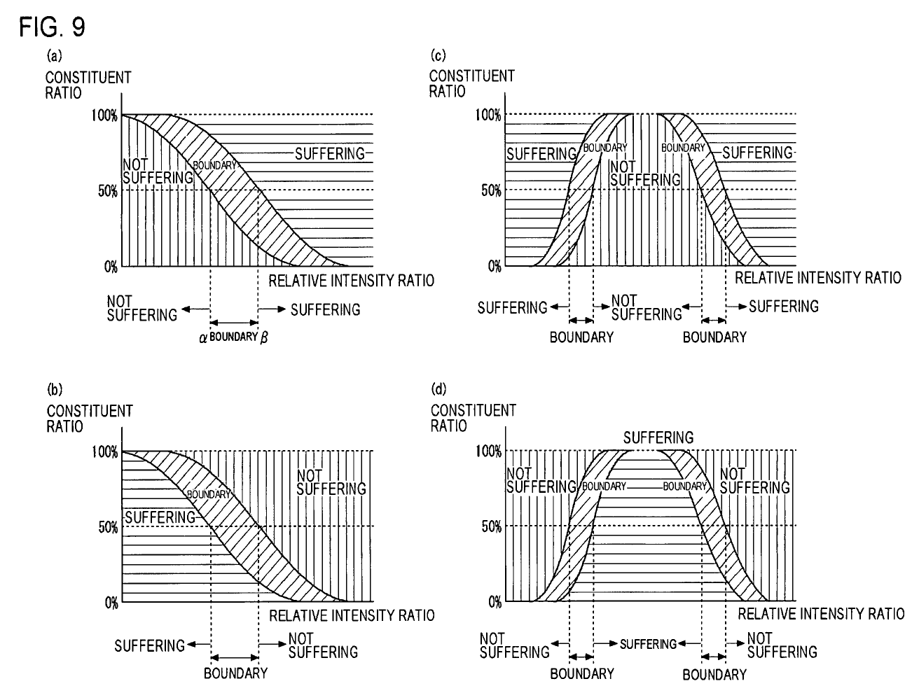

FIG. 9 is a view showing still another example of a data structure of the relationship data storing unit 35. Here, the relationship between the relative intensity ratio and the constituent ratio can be classified into patterns similar to those shown in FIG. 8; however, the method of inferring the possibility of non-suffering and suffering is different from the one shown in FIG. 8. For example, it is inferred to be not suffering when the possibility of not suffering exceeds a predetermined constituent ratio, while it is inferred to be suffering when the possibility of suffering exceeds a predetermined constituent ratio; and it is inferred to be a boundary when neither of the above holds. Here, assuming that the predetermined constituent ratio is 50%, for example, in the pattern shown in FIG. 9(a), the possibility of not suffering is about 50% when the relative intensity ratio is α. Therefore, the test subject is inferred to be not suffering when the relative intensity ratio is between 0 to α. Also, in this case, the possibility of suffering is about 50% when the relative intensity ratio is β. Therefore, the test subject is inferred to be suffering when the relative intensity ratio is β or above.

According to the diagnosis supporting system 10 of this embodiment, a desired protein marker is not adsorbed and captured with the use of a proper substance having an affinity, but a desired component is specified and the character thereof is extracted by letting the sample flow in the flow passageway for separation to separate the sample into plural components and making the information on each component corresponding to the movement parameter, so that the chip for separation of the sample is not dependent on a specific disease, and the detection of various diseases can be carried out with one kind of a chip. The chip 12 itself is used in common for the inference of the possibility of suffering from plural diseases, and the process of the diagnosis supporting system 10 is made different suitably in accordance with the disease, so that the possibility of suffering from various diseases can be inferred with a high general usability based on one diagnosis data.

### Second Embodiment

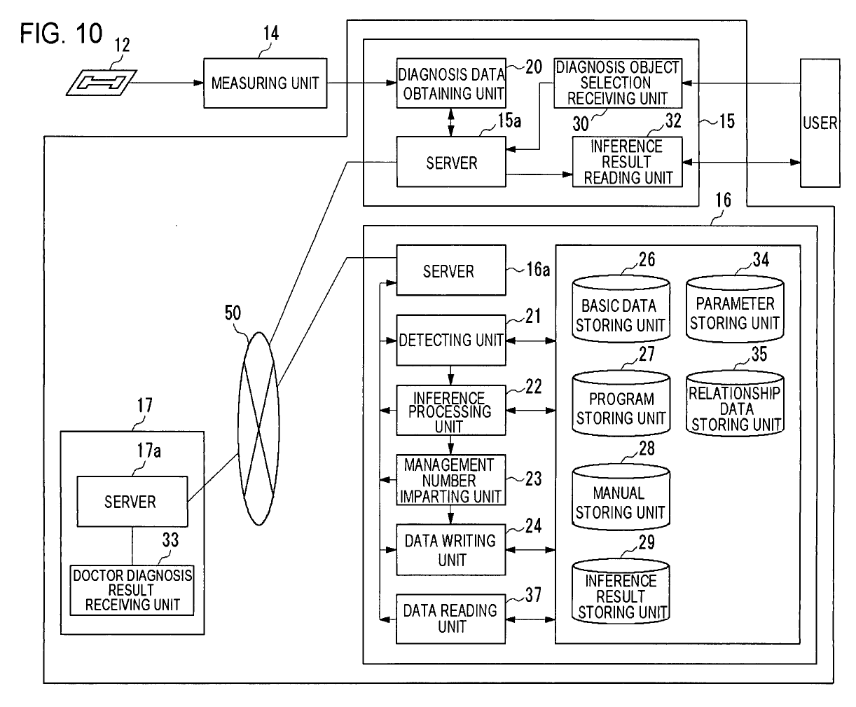

FIG. 10 is a block diagram showing a diagnosis supporting system 10 in the second embodiment of the present invention.

In this embodiment, the diagnosis supporting system 10 includes a measurement side system 15, an inference processing system 16, a hospital system 17, and a network 50 connecting these. Here, the measurement side system 15 includes a diagnosis data obtaining unit 20, a diagnosis object selection receiving unit 30, and an inference result reading unit 32, and sends and receives data to and from the inference processing system 16 via the network 50 with the use of a server 15a. The inference processing system 16 includes a detecting unit 21, an inference processing unit 22, a management number imparting unit 23, a data writing unit 24, a database 25, and a data reading unit 37, and sends and receives data to and from the measurement side system 15 and the hospital system 17 via the network 50 with the use of a server 16a. The data reading unit 37 reads various data out from the database 25. The hospital system 17 includes a doctor diagnosis result receiving unit 33, and sends and receives data to and from the inference processing system 16 via the network 50 with the use of a server 17a. In this embodiment, constituent elements similar to those of the first embodiment are denoted with similar symbols, and the description thereof will be suitably omitted. In this embodiment also, the measuring unit 14 is an UV spectroanalysis device.

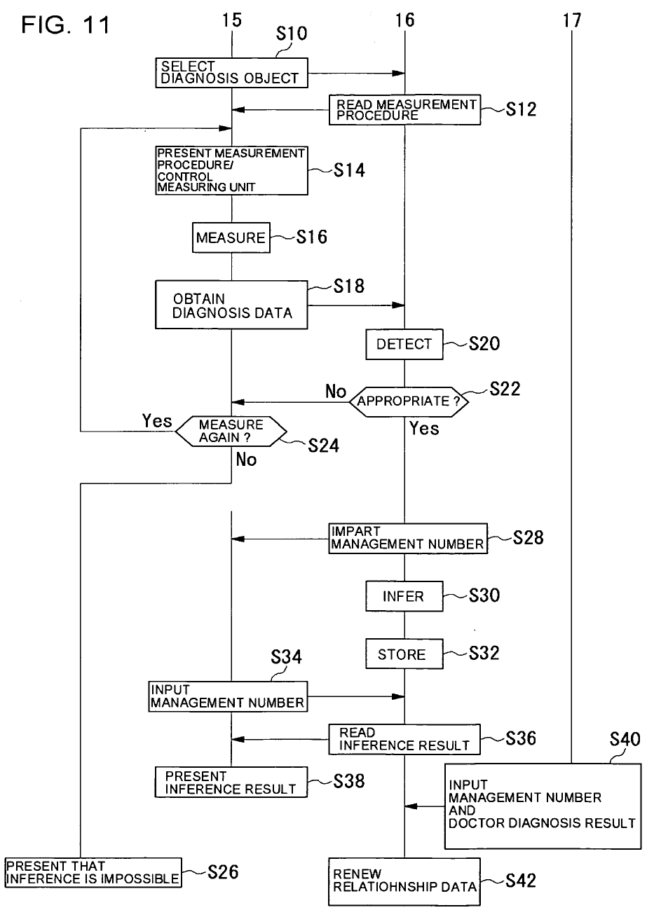

FIG. 11 is a flowchart showing a processing procedure in the measurement side system 15, the inference processing system 16, and the hospital system 17 in this embodiment. Hereafter, description will be made with reference also to FIG. 10.

First, in the measurement side system 15, when the user selects a disease name as an object of diagnosis from the diagnosis object selection receiving unit 30, that information is transmitted to the inference processing system 16 (S10). In the inference processing system 16, the data reading unit 37 reads a measurement procedure out from the manual storing unit 28, and transmits the measurement procedure to the measurement side system 15 via the server 16a (S12). In the measurement side system 15, the measurement procedure is presented to the user (S14). In the step 12, a program for controlling the measuring unit 14 may be read out, and in this case, the diagnosis data obtaining unit 20 controls the measuring unit 14 in accordance with the controlling program. In the measuring unit 14, when the character of the components in the sample on the chip 12 are measured (S16), the diagnosis data obtaining unit 20 obtains, as diagnosis data, the character of the components in the sample respectively in correspondence with the movement parameters (S18). The diagnosis data are transmitted to the inference processing system 16.

The detecting unit 21 detects characteristic components or the like by making reference to the parameter storing unit 34 (S20). Here, the detecting unit determines whether the diagnosis data are appropriate or not (S22). Whether the diagnosis data are appropriate or not can be determined, for example, based on the character of the marker components that express themselves irrespective of the presence or absence of a specific disease. It is determined by whether the character of the marker components are within a predetermined range or not, or the like. When the character of these components are below a predetermined range, there is a fear that the concentration of the sample is too low to grasp the character of the characteristic components correctly. Also, when they are above a predetermined range such as when the character of these components are saturated, there is a fear that the character of the characteristic components or the like cannot be grasped correctly, making it impossible to carry out an appropriate inference. For this reason, when the diagnosis data are not appropriate in the step 22 (No in S22), the measurement side system 15 is notified of that fact, and the measurement side system 15 makes an inquiry to the user as to whether the measurement is to be carried out again (S24). When the user wishes to carry out the measurement again (Yes in S24), the procedure returns to the step 14, where the measurement is carried out again by presenting the measurement information. On the other hand, when the user does not wish to carry out the measurement again in the step 24 (No in S24), the measurement side system 15 presents the fact that the inference is impossible (S26), and ends the diagnosis process. When the diagnosis data are appropriate in the step 22 (Yes in S22), the management number imparting unit 23 imparts a management number to the diagnosis data, and informs the measurement side system 14 of the management number (S28). The imparting and informing of the management number may be carried out when the diagnosis data obtaining unit 20 has obtained the diagnosis data. At this time, the data writing unit 24 can store the diagnosis data in correspondence with the management number in the basic data storing unit 26. Subsequently, the inference processing unit 22 infers the possibility of suffering by making reference to the relationship data storing unit 35 (S30). The data writing unit 24 stores the inference result obtained by the inference processing unit 22 in correspondence with the management number in the inference result storing unit 29 (S32).

When the user requests for reading-out of an inference result by inputting a management number in the measurement side system 15 (S34), the inference result reading unit 32 reads a corresponding inference result out from the inference result storing unit 29 using the management number as a key (S36), and the inference result is presented to the user in the measurement side system 15 (S38).

Here, for example, when the test subject that has received an inference result by the diagnosis supporting system 10 receives a diagnosis of a doctor, the management number and the doctor diagnosis result are input from the doctor diagnosis result receiving unit 33 in the hospital system 17 (S40). In the inference processing system 16, the data writing unit 24 reads the inference result stored in the inference result storing unit 29 using the management number as a key, and renews the relationship data storing unit 35 by making reference to the doctor diagnosis result (S42).

In this embodiment, the measurement side system 15 may be disposed to be integral with the hospital system 17, and may be placed at a clinical place of a hospital or the like. In this case, at the clinical place, a minute amount of a sample is collected from a body fluid such as blood collected from a test subject, and a protein component is separated on a biochip including a flow passageway for separation that separates the sample in accordance with the nature. With respect to these components, the character is measured in the measuring unit 14 to obtain the diagnosis data.

In order to infer the test subject's possibility of suffering from a disease, one has to make reference to various data, and also an analysis takes time. For this reason, a system can be constructed in which only the collection and measurement of a sample is carried out at the clinical place, and an analysis for the inference may be carried out at an analysis center which is separate from the clinical place. By this, the inference of suffering can be made without placing a high-speed device for analysis at each clinical place, so that an inference result of the test subject's possibility of suffering can be obtained even at a clinical place that is located at a distant place.

According to the diagnosis supporting system 10 in this embodiment, even if the measurement of a sample is carried out at a distant place, the diagnosis data can be transmitted to the inference processing system 16 via the network 50, so that the possibility of suffering from various diseases can be inferred speedily.

### Third Embodiment

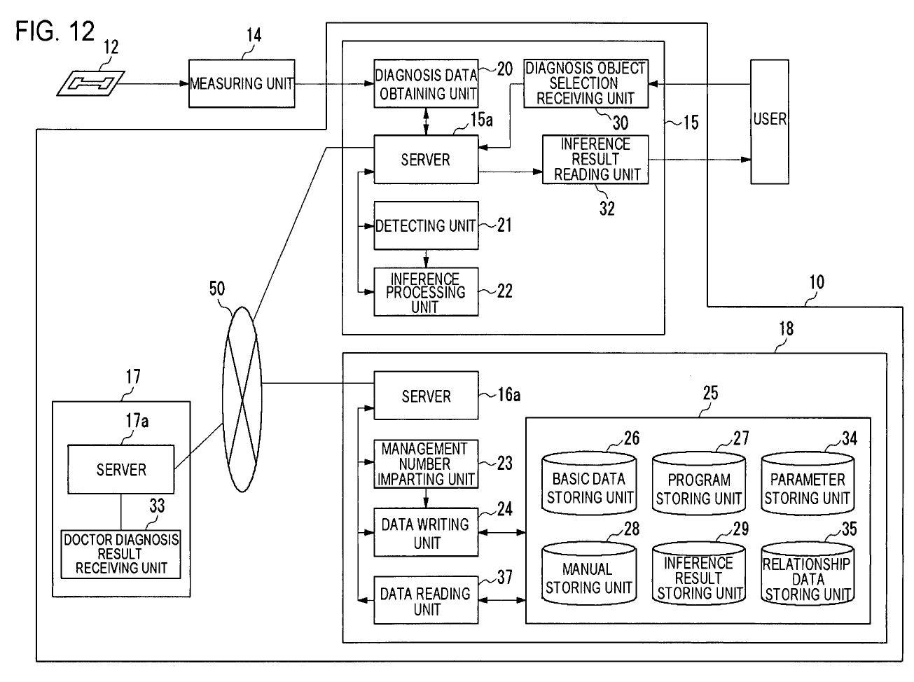

FIG. 12 is a block diagram showing a diagnosis supporting system 10 in the third embodiment of the present invention.

In this embodiment, the diagnosis supporting system 10 includes a measurement side system 15, a management system 18, a hospital system 17, and a network 50 connecting these. Here, the third embodiment is different from the second embodiment in that the detecting unit 21 and the inference processing unit 22 are included in the measurement side system 15. The management system 18 includes a management number imparting unit 23, a data writing unit 24, a database 25, and a data reading unit 37. In this embodiment, constituent elements similar to those of the first and second embodiments are denoted with similar symbols, and the description thereof will be suitably omitted. In this embodiment also, the measuring unit 14 is an UV spectroanalysis device.

In this embodiment, the management system 18 is disposed to be accessible from a plurality of measurement side systems 15. By this, various data can be shared in common by the plural measurement side systems 15, and more basic data or the like can be accumulated in the database 25, so that the possibility of suffering can be inferred with better precision.

### Fourth Embodiment

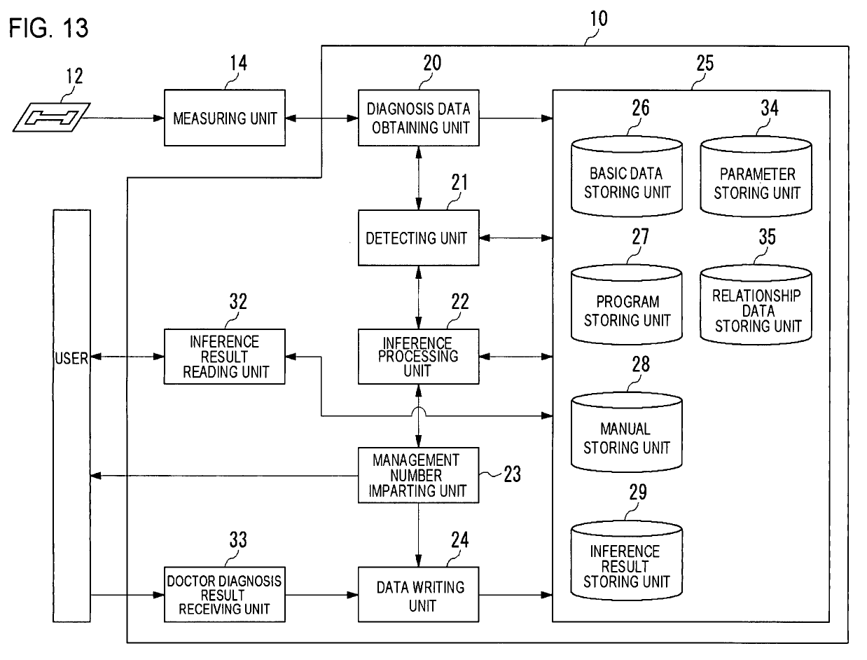

FIG. 13 is a block diagram showing a diagnosis supporting system 10 in the fourth embodiment of the present invention.

In this embodiment, the diagnosis supporting system 10 is different from the diagnosis supporting system 10 of the first embodiment shown in FIG. 1 in that it does not have a diagnosis object selection receiving unit 30. In this embodiment, constituent elements similar to those of the first embodiment are denoted with similar symbols, and the description thereof will be suitably omitted. In this embodiment also, the measuring unit 14 is an UV spectroanalysis device. In this embodiment, the diagnosis supporting system 10 infers the possibility of suffering respectively for a plurality of diseases based on the diagnosis data obtained from one test subject.

In this embodiment, the detecting unit 21, for each disease, reads the movement parameter of a corresponding characteristic component out from the parameter storing unit 34, and detects the characteristic component respectively from the diagnosis data by making reference to the movement parameter. The inference processing unit 22, for each disease, reads the relationship data out from the relationship data storing unit 35, and infers the possibility of suffering from a specific disease of a test subject that has offered the sample by making reference to the relationship data.

In this manner, according to the diagnosis supporting system 10 in this embodiment, the possibility of suffering can be inferred for plural diseases from one diagnosis data, so that the possibility of suffering from various diseases, for which the movement parameters of the characteristic components and the relationship data are stored in the database, can be collectively inferred.

### Fifth Embodiment

In the above first to fourth embodiments, description has been given by raising as an example a case in which the measuring unit 14 is an UV spectroanalysis device. This embodiment is different from the first to fourth embodiments in that the measuring unit 14 is a mass spectrometry device. In this embodiment also, the diagnosis supporting system 10 has a construction similar to that shown in the first to fourth embodiments. In this embodiment, the diagnosis data obtaining unit 20 obtains diagnosis data in which a movement parameter reflecting a movement speed of each component in letting the sample flow through the flow passageway for separation 112 of the chip 12 and separating the sample into plural components in accordance with a difference of the movement speed, a nature parameter showing nature of each component in classifying each component further into plural components in accordance with the nature, and a character of each component are in correspondence. Here, the movement parameter is a period of time of movement of each component for a constant distance. Also, the nature parameter is the molecular weight of each fragment when the plural components separated in the chip 12 are ionized. Also, the character of a component is a data value showing an amount of presence of each fragment.

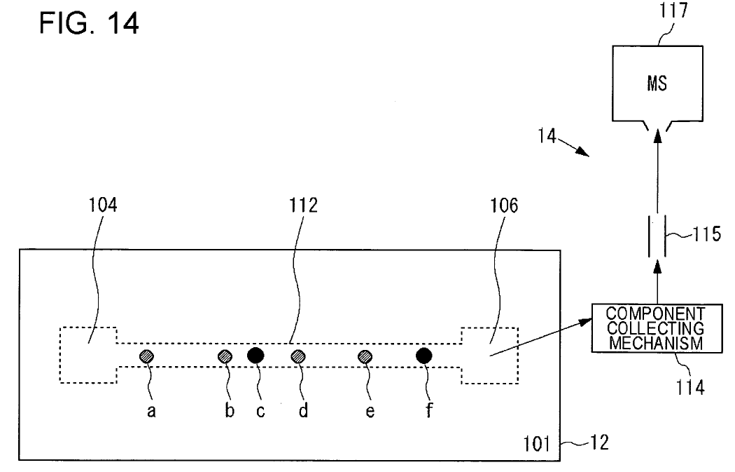

FIG. 14 is a perspective view showing the chip 12 and the measuring unit 14 in this embodiment. The chip 12 is similar to the one described in the first embodiment. The measuring unit 14 is an electrospray ionization mass spectrometry device (ESIMS). The measuring unit 14 has a component collection mechanism 114, an electrospray tube 115, and a mass spectrometry unit 117. The component collection mechanism 114 collects components from the sample collecting unit 106 of the chip 12 for every constant period of time, and introduces the components to the electrospray tube 115. A high voltage is applied to the tip end of the electrospray tube 115, and the components are ionized to be introduced into the mass spectrometry unit 117 by spraying the components from the electrospray tube 115. The components introduced into the mass spectrometry unit 117 are separated into plural fragments in accordance with the mass and electric charge of the ion, so as to be detected. By this, each component can be separated in accordance with the molecular weight. The measuring unit 14 measures the mass spectrometry data of each fragment. The diagnosis data obtaining unit 20 obtains the mass spectrometry data of each component in correspondence with the movement parameter. Here, the movement parameter is a period of time in which each component reaches the sample collecting unit 106. The movement parameter can be detected based on a timing by which the component collection mechanism 114 collects each component from the sample collecting unit 106.

In this embodiment, the parameter storing unit 34, for each of the plural diseases, stores the movement parameters and the nature parameter of plural components constituting an index in detecting from the diagnosis data the characteristic component for use in the inference of the possibility of suffering for the respective disease. The detecting unit 21 reads the movement parameter and the nature parameter of the characteristic component out from the parameter storing unit 34, and detects the characteristic component from the diagnosis data based on these parameters and the movement parameters and the nature parameter of the diagnosis data.

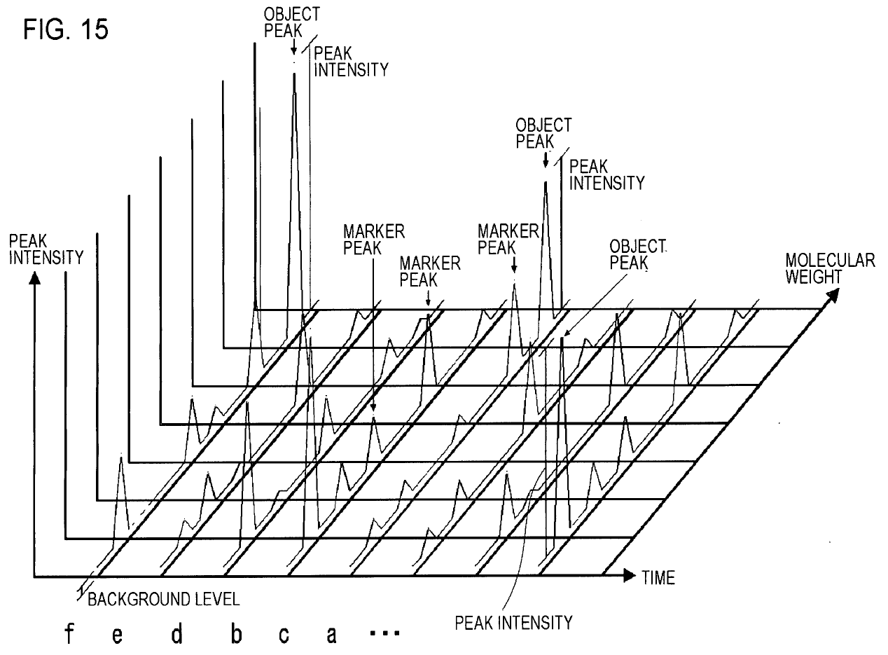

FIG. 15 is a view showing one example of the diagnosis data obtained by the diagnosis data obtaining unit 20. Here, the relationship among the period of time until reaching the sample collecting unit 106, the molecular weight, and the peak intensity of each component defined by these is shown. For example, the sample separated as the component f in FIG. 14 is further separated into plural components in accordance with the molecular weight.

In this embodiment, a mass spectrometry pattern is obtained for each component separated by the flow passageway for separation 112 of the chip 12, so that the possibility of suffering from various diseases can be inferred more correctly by comparing this mass spectrometry pattern. For example, when separation is carried out in the flow passageway for separation 112 in accordance with the molecular size of the component, the time axis will be one that reflects the molecular size of each component. Therefore, a map of peak intensity of the component specified by the difference in the molecular size and the difference in the molecular weight can be formed. By comparing the obtained map, the possibility of suffering from the disease can be inferred speedily.

As described above, in this embodiment, by making reference to the character of the component specified by plural parameters, the possibility of suffering from plural diseases can be inferred with better precision and in detail.

Here, the constituent elements of the diagnosis supporting system 10 in the above first to fifth embodiments can be any combination thereof, and can be constructed to be connected suitably via the network 50. Also, the measuring unit 14 can be disposed to be integral with any of the constituent elements of the diagnosis supporting system 10. For example, the measuring unit 14, the diagnosis data obtaining unit 20, the detecting unit 21, the inference processing unit 22, and the database 25 may be integrally constructed. Also, the measuring unit 14 and the diagnosis data obtaining unit 20 may be integrally constructed, and may be constructed to be connected to the inference processing unit 22 and the database 25 via the network 50. Further, the diagnosis data obtaining unit 20, the detecting unit 21, the inference processing unit 22, and the database 25 can be constructed to be each placed at a physically distant position and connected via the network.

Also, in order to detect the movement parameter of each component separated by the chip 12, a plurality of flow passageways 112 for separation can be formed in parallel on the chip 12, and a marker agent can be introduced into one flow passageway for separation and let to move through the flow passageway for separation 112 simultaneously with a sample collected from a test subject, so as to detect the movement parameter of each component by the position of the marker agent.

Also, in the above embodiments, the chip 12 can be constructed to separate the sample not only by the molecular size but also in accordance with other character such as an isoelectric point that the sample such as a protein generally has.

1-16. (canceled)

17. A diagnosis supporting system that infers a possibility that a test subject is suffering from a disease based on a sample collected from said test subject, said diagnosis supporting system comprising:
a diagnosis data obtaining unit that obtains diagnosis data in which a movement parameter reflecting a movement speed of each component in letting the sample move in a predetermined region and separating the sample into plural components in accordance with a difference of the movement speed and a character of each component are in correspondence;
a parameter storing unit that stores said movement parameter of a characteristic component that characteristically shows suffering from a specific disease and said movement parameter of a marker component that is detected irrespective of suffering from said specific disease, in correspondence with the disease;
a relationship data storing unit that stores relationship data showing a relationship between said character of said characteristic component and a possibility of suffering from said specific disease;
a detecting unit that reads said movement parameter of said characteristic component out from said parameter storing unit and detects said characteristic component from said diagnosis data based on the movement parameter and said movement parameter of said diagnosis data; and
an inference processing unit that reads said relationship data out from said relationship data storing unit and infers the possibility that said test subject is suffering from said specific disease based on said character of said characteristic component of said diagnosis data by making reference to the relationship data,
wherein said detecting unit detects a corresponding marker component from said diagnosis data by making reference to said movement parameter of said marker component and determines whether said characteristic of the marker component is appropriate or not and, when it is appropriate, detects said characteristic component and, when it is not appropriate, prompts a user to obtain a sample again.

18. The diagnosis supporting system of claim 17, wherein
said parameter storing unit stores a plurality of said movement parameters respectively in correspondence with the plural components, in correspondence with said disease, and
said detecting unit detects said characteristic component based on a mutual relationship of plurality of said movement parameters.

19. The diagnosis supporting system of claim 17 or 18, wherein
said parameter storing unit stores said movement parameter of said characteristic component for each of a plurality of diseases, and
said detecting unit, for each disease, reads said movement parameter of a corresponding characteristic component out from said parameter storing unit, and respectively detects said characteristic component from said diagnosis data by making reference to the movement parameter.

20. The diagnosis supporting system of any one of claims 17 or 18, wherein
said parameter storing unit stores said movement parameter of said characteristic component for each of a plurality of diseases,
said diagnosis data obtaining unit receives selection of a disease as an object of diagnosis together with said diagnosis data, and
said detecting unit, in accordance with the selection of a disease received by said diagnosis data obtaining unit, reads said movement parameter of a corresponding characteristic component out from said parameter storing unit, and detects said characteristic component from said diagnosis data by making reference to the movement parameter.

21. The diagnosis supporting system of any one of claims 17 or 18, wherein said character is an amount of change of light when light of a predetermined wavelength is radiated onto each of said separated components.

22. A diagnosis supporting system that infers a possibility that a test subject is suffering from a disease based on a sample collected from said test subject, said diagnosis supporting system comprising:
a diagnosis data obtaining unit that obtains diagnosis data in which a movement parameter reflecting a movement speed of each component in letting the sample move in a predetermined region and separating the sample into plural components in accordance with a difference of the movement speed, a nature parameter showing nature of each component in classifying each component further into plural components in accordance with the nature, and a character of each component are in correspondence;
a parameter storing unit that stores said movement parameter and said nature parameter of a characteristic component characteristically showing a state of suffering from a specific disease in correspondence with the disease;
a relationship data storing unit that stores relationship data showing a relationship between said character of said characteristic component and a possibility of suffering from said specific disease;
a detecting unit that reads said movement parameter and said nature parameter of said characteristic component out from said parameter storing unit and detects said characteristic component from said diagnosis data based on these parameters and said movement parameter and said nature parameter of said diagnosis data; and
an inference processing unit that reads said relationship data out from said relationship data storing unit and infers the possibility that said test subject is suffering from said specific disease based on said character of said characteristic component of said diagnosis data by making reference to the relationship data.

23. A diagnosis supporting system that infers a possibility that a test subject is suffering from a disease based on a sample collected from said test subject, said diagnosis supporting system comprising:
a diagnosis data obtaining unit that obtains diagnosis data in which a movement parameter when the sample is separated into plural components by letting the sample move in a predetermined region, a nature parameter showing nature of each component in classifying each component further into plural components in accordance with the nature, and a character of each component are in correspondence;
a parameter storing unit that stores said movement parameter and said nature parameter of a characteristic component characteristically showing a state of suffering from a specific disease in correspondence with the disease;
a relationship data storing unit that stores relationship data showing a relationship between said character of said characteristic component and a possibility of suffering from said specific disease;
a detecting unit that reads said movement parameter and said nature parameter of said characteristic component out from said parameter storing unit and detects said characteristic component from said diagnosis data based on these parameters and said movement parameter and said nature parameter of said diagnosis data; and
an inference processing unit that reads said relationship data out from said relationship data storing unit and infers the possibility that said test subject is suffering from said specific disease based on said character of said characteristic component of said diagnosis data by making reference to the relationship data.

24. The diagnosis supporting system of claim 22 or 23, wherein
said parameter storing unit stores a plurality of said movement parameters and said nature parameter respectively in correspondence with the plural components, in correspondence with said disease, and
said detecting unit detects said characteristic component from said diagnosis data based on a mutual relationship of said plurality of movement parameters and said nature parameter.

25. The diagnosis supporting system of any one of claims 22 or 23, wherein
said parameter storing unit stores said movement parameter and said nature parameter of said characteristic component for each of a plurality of diseases, and
said detecting unit, for each disease, reads said movement parameter and said nature parameter out from said parameter storing unit, and respectively detects said characteristic component from said diagnosis data by making reference to the movement parameter and said nature parameter.

26. The diagnosis supporting system of any one of claims 22 or 23, wherein
said parameter storing unit stores said movement parameter and said reference parameter of said characteristic component for each of a plurality of diseases,
said diagnosis data obtaining unit receives selection of a disease as an object of diagnosis together with said diagnosis data, and
said detecting unit, in accordance with the selection of a disease received by said diagnosis data obtaining unit, reads said movement parameter and said nature parameter of a corresponding characteristic component out from said parameter storing unit, and detects said characteristic component from said diagnosis data by making reference to the movement parameter and the nature parameter.

27. The diagnosis supporting system of claim 19, wherein
said relationship data storing unit stores said relationship data for each of a plurality of diseases, and
said inference processing unit, for each disease, reads said relationship data out from said relationship data storing unit and infers the possibility of said test subject's suffering from said specific disease by making reference to the relationship data.

28. The diagnosis supporting system of claim 19, wherein
said relationship data storing unit stores said relationship data of said characteristic component for each of a plurality of diseases, and
said inference processing unit, in accordance with the selection of a disease received by said diagnosis data obtaining unit, reads said relationship data of a corresponding disease and infers the possibility of said test subject's suffering from said specific disease by making reference to the relationship data.

29. The diagnosis supporting system of any one of claims 17 or 18, wherein said character is a data value showing an amount of presence of a specific substance in said component, and
said relationship data storing unit stores relationship data showing a relationship between a character function having said data value as a variable and said possibility of suffering.

30. The diagnosis supporting system of any one of claims 17 or 18,
further comprising a procedure storing unit that stores a procedure of obtaining said diagnosis data for each of a plurality of diseases, and
wherein said diagnosis data obtaining unit receives selection of a disease as an object of diagnosis before obtaining said diagnosis data, and reads said obtaining procedure related to a corresponding disease in accordance with the selection out from said procedure storing unit for presentation.

31. The diagnosis supporting system of any one of claims 17 or 18, further comprising:
a diagnosis data storing unit that stores said diagnosis data obtained by said diagnosis data obtaining unit in correspondence with a management number;
an inference result reading unit that outputs an inference result by said inference processing unit in correspondence with said management number;
a doctor diagnosis result receiving unit that receives a diagnosis result of a doctor on a specific disease together with said management number; and
a relationship data renewing unit that reads the corresponding diagnosis data out from said diagnosis data storing unit using said management number as a key, and renews said relationship data storing unit by making reference to said character of said characteristic component of the corresponding diagnosis data and the diagnosis result of said doctor.

32. The diagnosis supporting system of any one of claims 17 or 18, wherein said predetermined region is a flow passageway for separation that is disposed on a chip.

33. The diagnosis supporting system of claim 25, wherein
said relationship data storing unit stores said relationship data for each of a plurality of diseases, and
said inference processing unit, for each disease, reads said relationship data out from said relationship data storing unit and infers the possibility of said test subject's suffering from said specific disease by making reference to the relationship data.

34. The diagnosis supporting system of claim 26, wherein
said relationship data storing unit stores said relationship data for each of a plurality of diseases, and
said inference processing unit, for each disease, reads said relationship data out from said relationship data storing unit and infers the possibility of said test subject's suffering from said specific disease by making reference to the relationship data.
